(19) United States
(12) Patent Application Publication
(10) Pub. No.: US 2016/0157776 A1
(43) Pub. Date: Jun. 9, 2016

(54) WEARABLE DEVICE FOR STRESS ASSESSMENT AND MANAGEMENT AND METHOD OF ITS USE

(71) Applicant: Xerox Corporation, Norwalk, CT (US)

(72) Inventors: Lalit K. MESTHA, Fairport, NY (US); Nischal Murthy PIRATLA, Fremont, CA (US); Xuejin WEN, Fairport, NY (US)

(21) Appl. No.: 14/563,142

(22) Filed: Dec. 8, 2014

(51) Int. Cl. A61B 5/00; A61B 5/117; A61B 5/024

(52) U.S. Cl. CPC A61B 5/4884; A61B 5/02427; A61B 5/02438; A61B 5/7278; A61B 5/486; A61B 5/7246; A61B 5/7405; A61B 5/742; A61B 5/117; A61B 2503/12; A61B 2562/0238

(57) ABSTRACT

Systems and methods for assessing and managing stress of a user is provided. The system includes a wearable device that can be worn by the user; the wearable device include a sensing device for generating at least one time-series signal by continuous sensing of light intensity of light signals. The time-series signal includes at least one continuous photoplethysmographic (PPG) signal having an LF and an HF component. The system also includes a stress assessment device to determine a stress level of the agent based on a processing of the PPG signal. The stress assessment device further includes a feedback device configured by the processor to provide a feedback including at least one remedial message to the agent based on the determined stress level of the agent.

### TECHNICAL FIELD

The presently disclosed embodiments relate to stress assessment and management systems and devices, and more particularly to an apparatus that can be worn onto or near an area of exposed skin, such as a wrist, of a subject/person being monitored for stress assessment and management.

### BACKGROUND

The human body experiences stress due to a wide range of physiological and psychological external stimuli. Stress enables an active physiological response of the body to the external stimuli in a timely fashion. However, an abnormal increase in stress may compromise long-term health and disrupt the body's ability to respond to events that require a quick physical response, such as quickly pulling a hand away from a hot flame.

In a call center environment, agents often experience stress when communicating with customers for various reasons. For example, agents may experience stress when dealing with irate customers, or when the agent's role is either in conflict or ambiguous. Agent role conflict occurs when an agent has conflicting objectives to meet, such as where the agent is evaluated on the number of calls answered in a day. However, the agent may be simultaneously expected to resolve each caller's query/concern, which may result in calls lasting longer and thus decreasing the number of calls answered in a day. Agent role ambiguity occurs when the agent is either unaware of an appropriate action for a customer query, or lacks sufficient information for resolving the query. For example, customer complaints are usually related to inherent issues with respect to a client's product or service, over which the agent has little or no control (e.g., outage in access to a website due to annual maintenance). In another example, the agent may not have enough information to resolve a customer concern (e.g., the troubleshooting manual does not cover a particular type of problem).

Various measures are traditionally applied in a call center to improve customer care, as well as each agent's efficiency and work satisfaction. A few examples of these measures include collection of data related to audio analysis of the call, agent-generated call summaries, customer-provided feedback, and interactive voice response (IVR) call routing. The collected data is manually analyzed, such as by a supervisor, to identify customer issues and agent performance areas that needs improvement. This data can also be used for helping the agent by either reducing the agent call flow or to provide relevant assistance to the agent. The time delay due to offline analyses of the collected data impeded or prevents the supervisor from effectively monitoring multiple calls in a live environment to ensure or enhance customer satisfaction. Additionally, the agent's stress level during a customer call is not taken into consideration to perform the above analyses. As a result, the related art fails to provide appropriate long-term remedial solutions for the agent to improve performance and work satisfaction.

### SUMMARY

In some situations, video recording of the session may be discouraged. It is therefore necessary to provide a reliable solution with a wearable device that may detect agent's stress level and provide feedback on an interactive basis between an agent and a customer based on the agent stress-level during a live customer call. While at the same time, the body worn device can be used to give indication to the agent that his/her physiological state is normal for resuming the job.

An exemplary embodiment of the present disclosure provides a system for assessing and managing stress of an agent while interacting with a customer. The system includes a wearable device configured to be worn by the agent. The wearable device includes a sensing device for generating at least one time-series signal by continuous sensing of light intensity of one or more light signals. The at least one time-series signal includes at least one continuous photoplethysmographic (PPG) signal including at least one low frequency (LF) component and at least one high frequency (HF) component. The system further includes a stress assessment device using a processor to determine a stress level of the agent based on a processing of the at least one PPG signal. The stress assessment device further includes a signal analysis device for receiving the at least one time-series signal from the wearable device; and processing the at least one time-series signal to generate the at least one continuous PPG signal. The signal analysis device is also configured to compute a ratio of the at least one LF component and the at least one HF component and determine the stress level of the agent based on the ratio exceeding a predefined stress threshold. The signal analysis module is further configured to generate a stress profile of the agent based on the determined stress level. The stress assessment device also includes a customer interaction analysis device configured to analyze customer interaction data of the agent based on a plurality of parameters; and determine stress-trigger points from the customer interaction data for the agent. The stress assessment device also includes a feedback device configured to provide a feedback comprising at least one remedial message to the agent based on the determined stress level of the agent.

Another exemplary embodiment of the present disclosure provides a method for assessing and managing stress of an agent while interacting with a customer. The method includes generating, by the sensing device of a wearable device, at least one time-series signal based on continuous sensing of light intensity of the one or more light signals. The agent wears the wearable device during the sensing. The at least one time-series signal includes at least one photoplethysmographic (PPG) signal including at least one low frequency (LF) component and at least one high frequency (HF) component. The method further includes determining, by a stress assessment device, a stress level of the agent based on a processing of the at least one PPG signal in real time by: processing, by a signal analysis device, the at least one time-series signal to generate the at least one continuous PPG signal; computing, by the signal analysis device, a ratio of the at least one LF component and the at least one HF component; determining, by the signal analysis device, the stress level of the agent based on the ratio exceeding a predefined stress threshold; and generating, by the signal analysis device, a stress profile of the agent based on the determined stress level. The method further includes providing, by a feedback device, a feedback to the agent based on the determined stress level of the agent in the real time.

Another exemplary embodiment of the present disclosure provides a system for assessing and managing stress of a user. The system includes a wearable device configured to be worn by the user. The wearable device includes a sensing device for generating at least one time-series signal by continuous sensing of light intensity of one or more light signals. The at least one time-series signal includes at least one continuous photoplethysmographic (PPG) signal including at least one low frequency (LF) component and at least one high frequency (HF) component. The system includes a stress assessment device using a processor to determine a stress level of the user based on a processing of the at least one PPG signal. The stress assessment device further includes a signal analysis device configured to process the at least one time-series signal to generate the at least one continuous PPG signal and compute a ratio of the at least one LF component and the at least one HF component. The signal analysis device is further configured to determine the stress level of the user based on the ratio exceeding a predefined stress threshold and generate a stress profile of the user based on the determined stress level. The stress assessment device further includes an interaction analysis device to analyze interaction data of the user based on a plurality of parameters and determine one or more stress-trigger points from the interaction data for the user. The stress assessment device further includes a feedback device to provide a feedback including at least one remedial message to the user based on the determined stress level of the user.

A further exemplary embodiment of the present disclosure provides a method for assessing and managing stress of a user. The method includes generating, by a sensing device of a wearable device, at least one time-series signal based on continuous sensing of light intensity of the one or more light signals. The user can wear the wearable device during the sensing. The at least one time-series signal includes at least one photoplethysmographic (PPG) signal including at least one low frequency (LF) component and at least one high frequency (HF) component. The method also includes determining, by a stress assessment device, a stress level of the agent based on a processing of the at least one PPG signal in real time by: processing, by a signal analysis device, the at least one time-series signal to generate the at least one continuous PPG signal; computing, by the signal analysis device, a ratio of the at least one LF component and the at least one HF component; determining, by the signal analysis device, the stress level of the user based on the ratio exceeding a predefined stress threshold; generating, by the signal analysis device, a stress profile of the user based on the determined stress level; analyzing, by an interaction analysis device, interaction data of the user base don a plurality of parameters; determining one or more stress-trigger points from the interaction data for the user; and providing, by a feedback device, a feedback to the agent based on the determined stress level of the user in the real time.

A yet another exemplary embodiment of the present disclosure provides a wearable device for generating feedback to an agent during communication with a customer. The wearable device includes a signal analysis device for determining a stress level of the agent based on the time-series signal received from the wearable device, the wearable device being configured to be worn onto or near an area of exposed skin of the agent. The wearable device can generate at least one time-series signal by continuous sensing of light intensity of one or more light signals. The at least one time-series signal may include at least one photoplethysmographic (PPG) signal. The system also includes a customer interaction analysis device for receiving data collected based communication between the agent and the customer, the customer interaction analysis device is configured to analyze customer interaction data of the agent and correlate the data with a determined stress-level over the predefined time interval. The system further includes a feedback device configured to generate feedback to the agent based on the determined stress-level exceeding a predefined stress threshold, the feedback including predefined suggestive messages based on the correlated data.

A further exemplary embodiment of the present disclosure provides a method for generating feedback to an agent during communication with a customer. The wearable device includes determining, by a signal analysis device, a stress level of the agent based on the time-series signal received from the wearable device. The wearable device being configured to be worn onto or near an area of exposed skin of the agent. The wearable device is configured to generate at least one time-series signal by continuous sensing of light intensity of one or more light signals, wherein the at least one time-series signal comprises at least one photoplethysmographic (PPG) signal including at least one low frequency (LF) component and at least one high frequency (HF) component. The method also includes receiving, by an interaction analysis device, data collected based communication between the agent and the customer, the customer interaction analysis device is configured to analyze customer interaction data of the agent and correlate the data with a determined stress-level over the predefined time interval. The method further includes generating, by a feedback device, feedback to the agent based on the determined stress-level exceeding a predefined stress threshold, the feedback including predefined suggestive messages based on the correlated data.

Other and further aspects and features of the disclosure will be evident from reading the following detailed description of the embodiments, which are intended to illustrate, not limit, the present disclosure.

### BRIEF DESCRIPTION OF THE DRAWINGS

FIGS. 1-3 illustrate exemplary network environments including a stress assessment device according to embodiments of the present disclosure;

FIGS. 4A-4C illustrate exemplary network environments including a stress assessment device according to embodiments of the present disclosure;

FIG. 5 is a block diagram illustrating various system elements of an exemplary wearable device according to an embodiment of the present disclosure;

FIG. 6 is a block diagram illustrating various system elements of an exemplary stress assessment device according to an embodiment of the present disclosure;

FIG. 7A is a graph depicting a time-series signal for a red LED;

FIG. 7B is a graph depicting a normalized time-series signal;

FIG. 8 is a graph depicting a filtered time-series signal;

FIG. 9 is a graph depicting a Power Spectral Density (PSD) of a PPG signal derived from the wearable device;

FIG. 10 is a table summarizing spectral components of various heart rate (HR) signals for explanatory purposes;

FIG. 11 is a block diagram illustrating various system elements of an exemplary wearable device according to another embodiment of the present disclosure; and

FIG. 12 is a flowchart illustrating an exemplary method for assessing and managing stress of an agent while interacting with a customer according to an embodiment of the present disclosure.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

### Non-Limiting Definitions:

In various embodiments of the present disclosure, definitions of one or more terms that will be used in the document are provided below.

A “video” is a time-varying sequence of images captured of a subject of interest using a video camera capable of acquiring a video signal over at least one data acquisition (imaging) channels. The video may also contain other components such as, audio, time reference signals, and the like.

A “time-series signal” refers to a time varying signal generated from images of the captured video. Time-series signals may be generated in real-time from a streaming video as in the case of continuous agent monitoring. The time-series signal may be obtained directly from the data acquisition channel of the video camera used to capture the video of the subject of interest. The time-series signal may be retrieved from a remote device such as a computer workstation over a wired or wireless network or obtained on a continuous basis from a video stream.

“Cardiac pulse” or “Photoplethysmographic (PPG) signal” is a pressure wave that is generated by the subject's heart (in systole) as the heart pushes a volume of blood into the arterial pathway. Arterial movement, as a result of this pressure wave, can be sensed by tactile and electronic methods. A frequency range of the cardiac pulse is the pulse rate measured over time, typically recorded in beats per minute (bpm) with upper and lower limits. The frequency range of the human cardiac pulse is between about 40 bpm to 240 bpm. A resting adult human, typically aged 18+ years has a heart rate of 60 to 100 bpm. For an adult athlete, the resting heart rate will be 40 to 60 bpm. Cardiac output, i.e., the volume of blood the heart can pump in one minute which is expressed in L/min (˜5.6 L/min for an adult human male and 4.9 L/min for an adult human female) and is proportional to heart rate.

A “wearable device” refers to a device that can be worn circumferentially onto or near an area of an exposed skin of the agent. The wearable device includes sensing device for sensing light signals.

### Exemplary Embodiments:

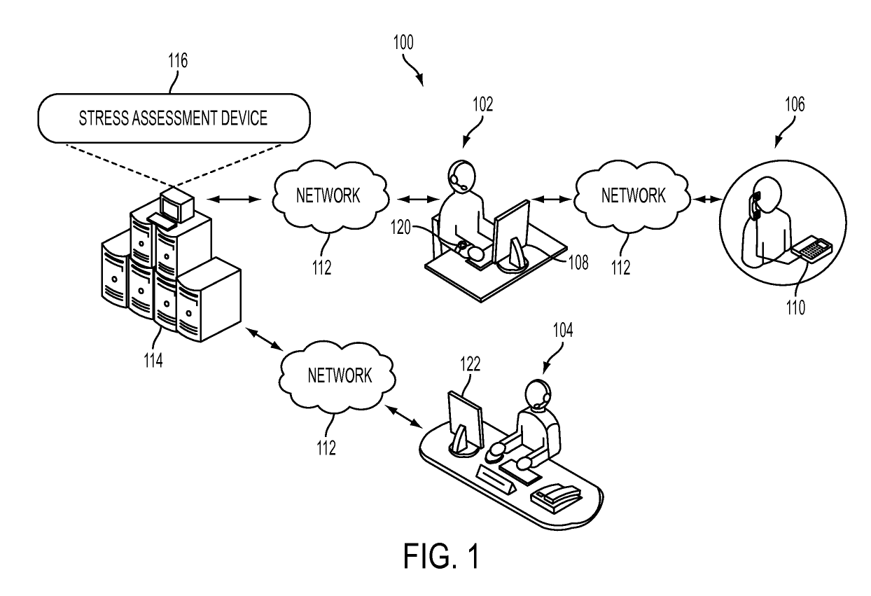

FIGS. 1-3 illustrate exemplary network environments that each includes a stress assessment device 116 and a wearable device 120, according to some exemplary embodiments of the present disclosure. The embodiments are disclosed in the context of network environments that represent a communication pathway for a call center to enhance interaction among an agent 102, a call center supervisor 104, and a customer 106. However, other embodiments can be applied in the context of other business scenarios involving interactions between different entities including customers, employees, colleagues, vendors, consultants, vehicle drivers, and so on. Examples of such scenarios include, but are not limited to, bank agents handling customer account workflows or related processes, hospital agents handling patient documents (such as in the context of new patients in emergency situations), healthcare professionals handling patient interactions in a tele-health environment, retail agents handling customer's return counters, teachers or students handling coursework, etc.

The agent 102 and the customer 106 may communicate with each other using an agent device 108 and a customer device 110, respectively, in different network environments. The agent device 108 may be implemented as any of a variety of computing devices, including, for example, a server, a desktop PC, a notebook, a workstation, a personal digital assistant (PDA), a mainframe computer, a mobile computing device, an internet appliance, and so on. The agent device 108 is configured to exchange at least one of text messages, audio interaction data (e.g., voice calls, recorded audio messages, etc.), and video interaction data (e.g., video calls, recorded video messages, etc.), or a combination of these with the customer device 110, or in any combination. Examples of the customer device 110 may include, but are not limited to, calling devices (e.g., a telephone, an internet phone, etc.), texting devices (e.g., a pager), or computing devices including those mentioned above. Further, the agent 102 may wear the wearable device 120 either while communicating with the customer 106 or when the agent 102 is on duty at work. The agent may experience stress while interacting with the customer 106.

The wearable device 120 can be worn onto or near to area of exposed skin, such as a wrist, waist, feet, neck, and so forth, of the agent 102. The size such as length, width, height, etc., and shape of the wearable device 120 may vary depending on an area for wearing the wearable device 120. Though not shown, but the wearable device 120 may be worn by any person whose stress need to be assessed and managed, for example, the wearable device 120 can be worn by vehicle drivers, babies in the ICU, employees working in the office, athletes, and so forth. The wearable device 120 may generate at least one time-series signal by continuous sensing of light intensity of one or more light signals. In some embodiments, the wearable device 504 may continuously sense the light intensity of one or more light signals with a sampling frequency of 3 Hz or greater. The at least one time-series signal may include at least one PPG signal, which is generated by the heart of the agent 102 as the heart pushes a volume of blood into the arterial pathway. The wearable device 120 may communicate the processed PPG signal to the stress assessment device 116 for further processing.

In a first exemplary network environment 100 (FIG. 1), the agent device 108 may be configured to interact directly with the customer device 110 via a network 112. The network 112 may be a wireless or a wired network, or a combination thereof. The network 112 may be a collection of individual networks, interconnected with each other and functioning as a single large network (e.g., the Internet or an intranet). Examples of the network 112 may include, but are not limited to, local area network (LAN), wide area network (WAN), a personal area network (PAN), a cable/telephone network, a satellite network, and so forth.

The agent device 108 may collect a variety of customer interaction data during communication with the customer device 110. For example, the agent device 108 may be installed with a known, related art or later developed interactive voice response (IVR) system (not shown). The IVR system interfaces with the customer device 110 before the customer 106 can interact with the agent 102 through various modes, such as text messages, audio interactions (e.g., voice calls, recorded audio messages, etc.), and video interactions (e.g., video calls, recorded video messages, etc.).

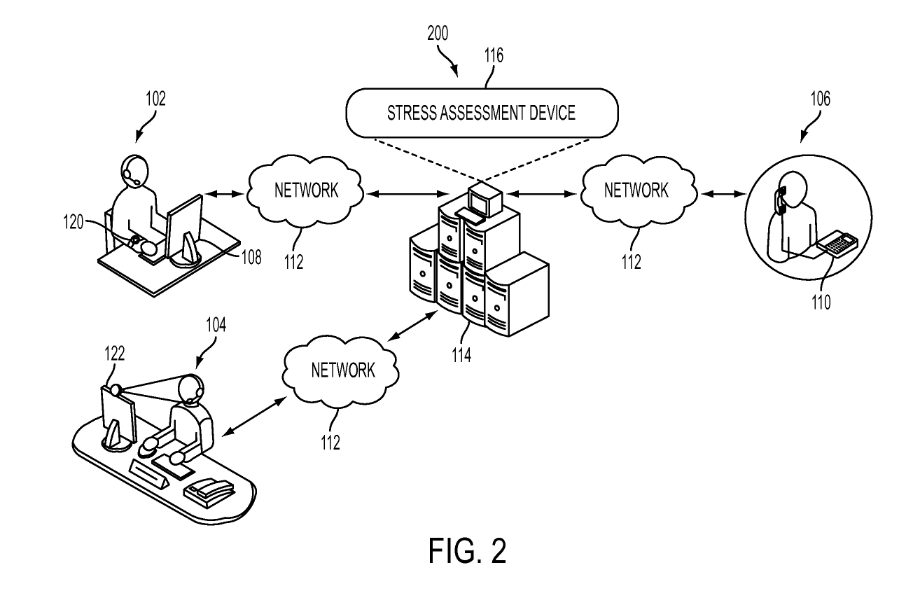

In a second exemplary network environment 200 (FIG. 2), the agent device 108 may be configured to interact with the customer device 110 via a server 114. The server 114 may connect the agent device 108 to the customer device 110 over the network 112. Optionally, the IVR system may be installed on the server 114 for interfacing with the customer device 110. The server 114 may be implemented as a specialized computing device implementing the embodiments. Alternatively, server 114 may be implemented any of a variety of computing devices including, for example, multiple networked servers (arranged in clusters or as a server farm), a mainframe, or so forth.

The customer 106 may submit voice inputs or dual tone multi-frequency (DTMF) tone inputs to the IVR system using the customer device 110 in response to prerecorded or dynamically generated audio messages in the IVR system. Subsequently, the customer 106 may be routed via the IVR system to the agent device 108 for interacting with the agent 102. However, other examples may include one or more agent devices configured to establish a direct communication with the customer devices for exchanging text messages, audio interaction data, and video interaction data without the IVR system. The agent device 108 may convey the customer interaction data including the text messages, the audio interaction data, and the video interaction data conducted between the customer 106 and the agent 102 (or the supervisor 104), agent-generated customer call summaries after communication with the customer 106, customer-provided feedback and customer's responses to the IVR audio messages, to the server 114. The agent device 108 is configured to provide agent identification data along with the customer interaction data to the server 114. Examples of the agent identification data include, but are not limited to, agent login ID, agent name, IP address of the agent device 108, and so on. In some embodiments, the agent device 108 may tag the agent identification data with the customer interaction data.

The server 114 includes the stress assessment device 116 for analyzing the customer interaction data and/or the received time-series signals received from the agent device 108 (FIG. 1) or the customer device 110 (FIG. 2). Along with the customer interaction data, the server 114 also receives the corresponding agent identification data from the agent device 108 and the time-series signals including the PPG signals from the wearable device 120. The stress assessment device 116 may process the received data and based on a correlation between the customer interaction data and the time-series data, a stress level of the agent may be determined.

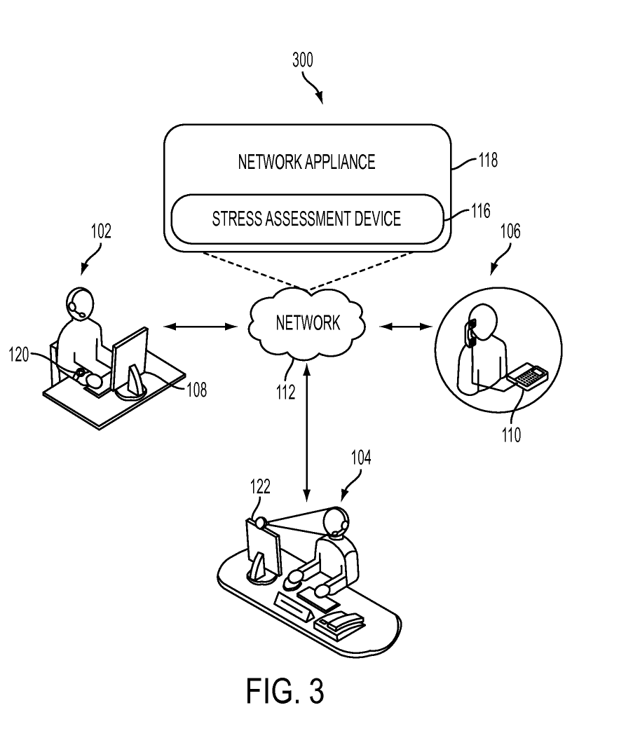

Similar to the network environment 100 (FIG. 1), a third exemplary network environment 300 (FIG. 3) may implement the agent device 108 to interact with the customer device 110 over the network 112. In one embodiment, the network 112 may be established using a network appliance 118 that may be integrated with the stress assessment device 116. In other embodiments, the network appliance 118 may be preconfigured or dynamically configured to include the stress assessment device 116 integrated with other devices. For example, the stress assessment device 116 may be integrated with the agent device 108. The agent device 108 may include a device (not shown) that enables the agent device 108 being introduced to the network appliance 118, thereby enabling the network appliance 118 to invoke the stress assessment device 116 as a service. Examples of the network appliance 118 include, but not limited to, a DSL modem, a wireless access point, a router, and a gateway for implementing the stress assessment device 116.

The stress assessment device 116 may represent any of a wide variety of devices that provide services for the network 112. The stress assessment device 116 may be implemented as a standalone and dedicated “black box” including specialized hardware with a processor and memory programmed with software, where the hardware is closely matched to the requirements and/or functionality of the software. The stress assessment device 116 may enhance or increase the functionality and/or capacity of the network 112 to which it is connected. The stress assessment device 116 may be configured, for example, to perform e-mail tasks, security tasks, network management tasks including IP address management, and other tasks. In some embodiments, the stress assessment device 116 is configured not to expose its operating system or operating code to an end user, and does not include related art I/O devices, such as a keyboard or display. The stress assessment device 116 of some embodiments may, however, include software, firmware or other resources that support remote administration and/or maintenance of the stress assessment device 116.

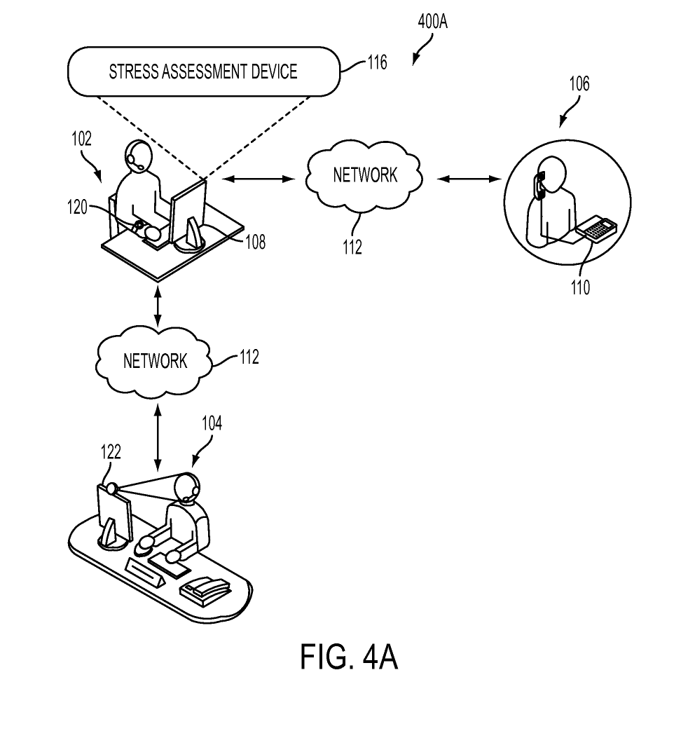

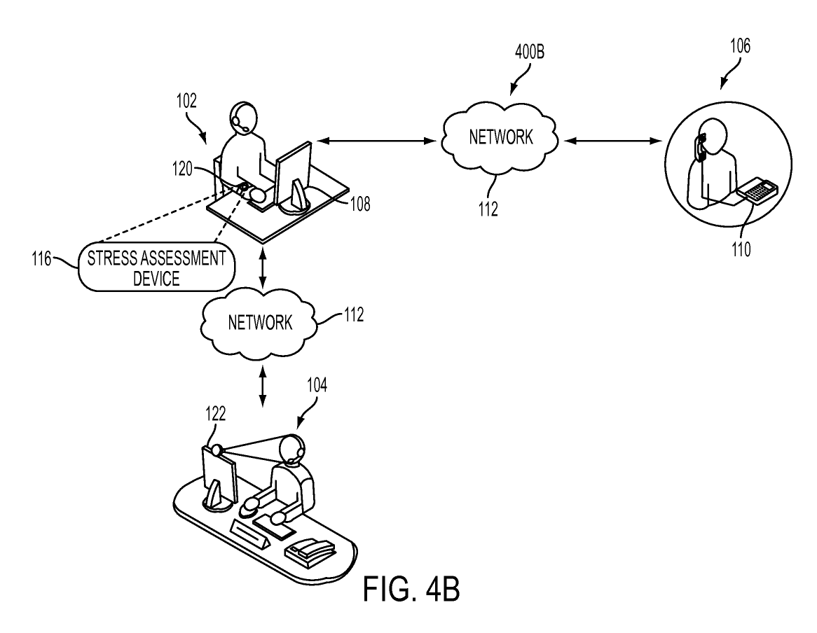

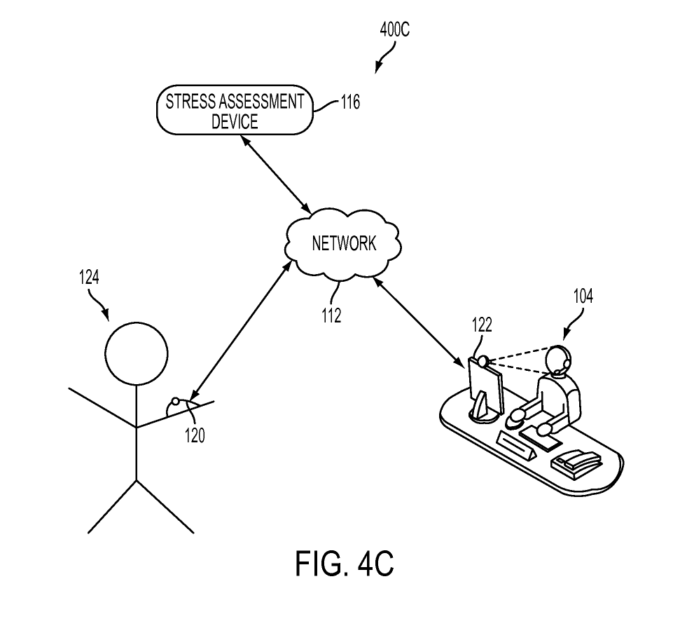

FIGS. 4A-4C illustrate exemplary network environments 400A-400D that each includes the stress assessment device 116 and the wearable device 120, according to some further exemplary embodiments of the present disclosure. As shown in the exemplary network environment 400A (FIG. 4A), the stress assessment device 116 may be integrated with, or installed on, the agent device 108 that directly communicates with the customer device 110 over the network 112. In further embodiments, as shown in exemplary network environment 400B (FIG. 4B), the stress assessment device 116 may be integrated with, or installed on, the wearable device 120. The stress assessment device 116, discussed below in greater detail, may be configured to estimate stress-levels of multiple agents, such as the agent 102, upon receiving their videos and generate feedback on real-time or at predetermined intervals to corresponding agents about the estimated stress-levels.

The stress assessment device 116 may also receive the time-series signal and PPG signal from the wearable device 120 for analysis. The wearable device 120 may be tied or worn on or near a region of an agent's body. The region can be a very small region of interest (ROI) which is in direct contact with the wearable device 120, where a photoplethysmograph (PPG) signal (described more fully below) of the agent 102 can be registered. Examples of the very small ROI may include wrist, thigh, waist, neck, ankle, feet, and hand or any suitable area where the wearable device 120 can be worn. The PPG signals are part of a time-series signals and may be generated by the agent's heart as the heart pushes the volume of blood into the arterial pathway.

The stress assessment device 116 can be configured to receive the time-series signals from the wearable device 120 and process the signals to estimate or determine a stress level of the agent 102. In some embodiments, the stress assessment device 116 is within the wearable device 120. Based on the stress level of the agent, the stress assessment device 116 may provide a real-time feedback to the agent device 108 or a supervisor device 122 associated with the supervisor 104 based on the agent stress exceeding a predefined threshold during a live customer interaction. The embodiments for a stress assessment device are intended to cover any and/or all devices capable of performing respective operations on the agent in a customer-interacting environment relevant to the applicable context.

The stress assessment device 116 analyzes the generated time-series signal to estimate/determine the stress-levels of the agent 102 “passively” through any known, related art or later developed non-contact mechanisms, in real-time while a customer-agent interaction is in progress, or alternatively, after a customer-agent interaction has ended. For example, heart rate variability (HRV) is a common measure that may be used for evaluating agent stress by determining a state of the autonomic nervous system (ANS) of the agent 102. The ANS is represented by the sympathetic nervous system (SNS) and the parasympathetic nervous system (PNS) of the agent 102. HRV is the beat-to-beat time variation in heartbeat and is modulated by changes in the balance between influences of the SNS and the PNS. Such changes occur based on the response of the agent's body to stress by releasing hormones, such as epinephrine and cortisol, which in turn lead to increase in heartbeat, tightening of muscles, and increase in blood pressure. HRV is also useful for diagnosis of various diseases and health conditions such as diabetic neuropathy, cardio vascular disease, myocardial infraction, fatigue, sleep problems, psychiatric disorders, and psychological disorders.

In some embodiments, the stress assessment device 116 extracts and analyses a ratio of low-frequency (LF) and high-frequency (HF) components of the integrated power spectrum of the generated time-series signal. When the LF/HF ratio is greater than a stress threshold value, for example, a value “1”, the agent's SNS is more dominant and hence indicates that the agent 102 is under stress. Accordingly, the stress assessment device 116 generates feedback to the agent device 108 (or the supervisor device 122), as identified by the agent identification data, so that the agent 102 or the supervisor 104 may take appropriate action to reduce the agent stress.

The stress assessment device 116 is also configured to generate a stress profile of the agent based on the determined stress level. For example, an increasing level of agent stress based on the increasing LF/HF ratio may be assessed with respect to multiple predefined stress thresholds to create the stress profile for each agent 102 in real-time. Whenever the LF/HF ratio exceeds each of the predefined stress thresholds, feedback may be generated by the stress assessment device 116. Such stress profiles of different agents (such as agent 108) may be used, such as by the supervisor 104, for various purposes. For example, the agent stress profiles may assist the supervisor 104 to identify a set of agents that may be appropriate for: (1) a particular customer concern or issue; (2) further training on particular customer concerns or issues; or (3) customer concerns or issues that need better training material for the agents. Such analyses of the time-series signal enables the integration of multiple sources of data, such as the customer's audio responses to the agent 102 or the IVR system; color or textural changes in the exposed skin area of the agent 102; to enhance diagnosis of HRV and interactions with other effects of ANS.

Further, the stress assessment device 116 can be configured to analyze the customer interaction data based on various parameters. Examples of these parameters may include, but are not limited to, key words (e.g., “hello”, “late”, “hurry”, etc.), generic terms of interest (e.g., 16-character alphanumeric customer ID, 10-digit phone number, etc.), sentiment-intensive words (e.g., “hate”, “irritate”, etc.), user selections on the IVR system to identify a broad topic of a customer-agent conversation, customer-agent voice-over that may be indicative of impatience and irritation for the customer 106, and other aspects of the customer-agent interaction.

In an embodiment, the stress assessment device 116 can correlate the analyzed customer interaction data for each parameter with the determined stress profile or stress for each agent 102 based on the agent identification data to identify stress-trigger points. The stress assessment device 116 can be configured to generate predefined suggestive remedial messages based on the identified stress-trigger points. The stress assessment device 116 may be configured to communicate, either automatically or upon request, the predefined remedial messages along with the feedback, or otherwise, to the corresponding agent device 108 and/or the supervisor device 122. The predefined suggestive remedial messages assist the agent 102 and the supervisor 104 in real time to undertake appropriate actions to reduce agent stress during live communication with the customer 106.

As shown in FIG. 4C, the network environment 400C includes a user 124 wearing the wearable device 120. The wearable device 120 may or may not include the stress assessment device 116. Examples of the user 124 include, but are not limited to, an employee, a baby being monitored in an intensive care unit, a vehicle driver, an athlete, and so forth. In such scenarios, the stress assessment device 116 may determine or as the stress and provide feedback either to the user 124 or to the supervisor 104. The feedback may be an audio message, video message, a text message, and combination of these including a remedial message or suggestion for the user 124 or the supervisor 104.

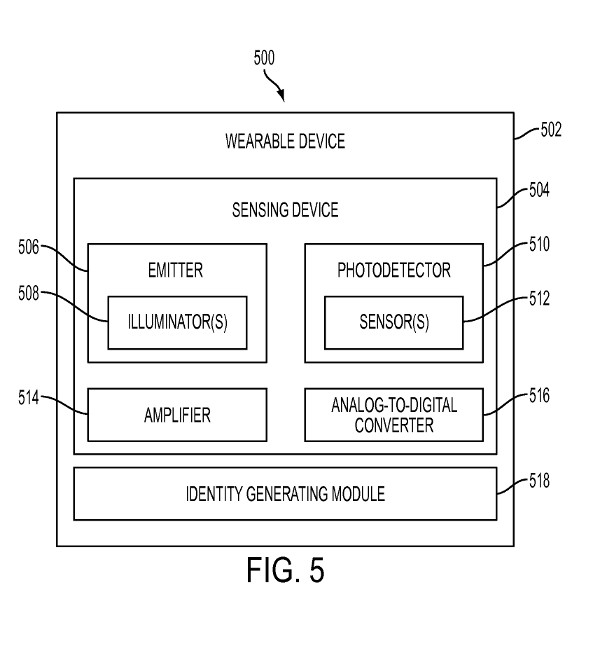

FIG. 5 is a block diagram 500 illustrating operational devices and features of the wearable device 120 as an exemplary wearable device 502 according to an embodiment of the present disclosure. The wearable device 502 may be worn by the agent 102 or the user 124 whose stress need to be assessed or managed. The wearable device 502 includes a sensing device 504 configured to continuously sense light intensity of one or more light signals, and generate at least one time-series signal based on the continuous sensing of the light intensity. In some embodiments, the sensing device 504 may continuously sense the light intensity of one or more light signals with a sampling frequency of 3 Hz or greater. The at least one time-series signal includes at least one photoplethysmographic (PPG) signal, which is generated by the agent's heart (or the user's heart) as the heart pushes a volume of blood into the arterial pathway. The continuous PPG signal includes at least one low frequency (LF) component and at least one high frequency (HF) component in the integrated power spectrum of the PPG signal over a predefined interval.

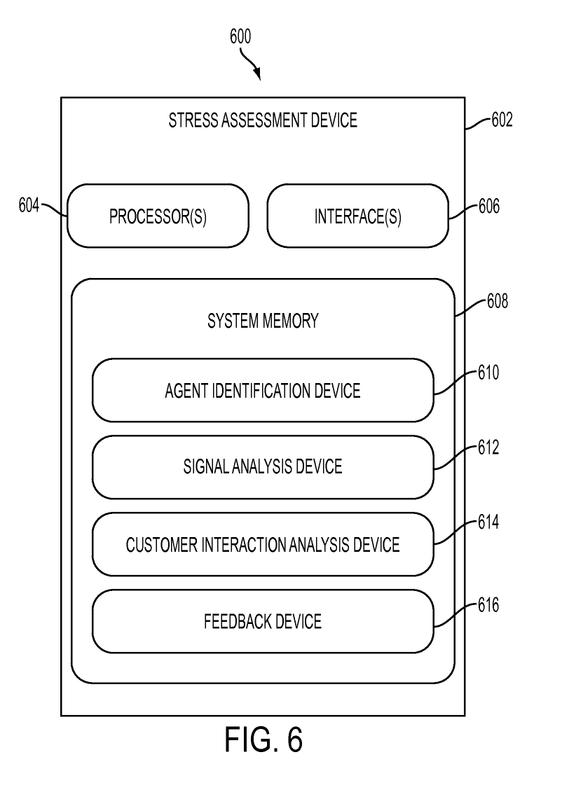

The sensing device 504 further includes at least one emitter 506 fixed to an inner surface of the wearable device 502. The sensing device 504 is capable of providing photoplethysmographic (PPG) signal or a cardiac pulse signal that is generated by the subject's (or agent's) heart as the heart pushes a volume of blood into the arterial pathway. The emitter 506 also includes at least one illuminator 508 (or illuminators) configured to emit light at a specified wavelength band. Further, the sensing device 504 also includes a photodetector 510 including at least one sensor 512. The photodetector 510 may be coupled to the at least one emitter 506. Though only one emitter 506 and one photodetector 510 is shown, a person ordinarily skilled in the art will appreciate that the wearable device 502 or the sensing device 504 may include more than one pair of emitter 506 and the photodetector 510. Further, the wearable device 502 may include more than one pair of emitter 506 and detector 510 (or photodetector 510) for improving the signal strength. In some embodiments, the emitter(s) 506 is set to emit light at a specified wavelength range centered on 660 nm because the absorbance of light in the red region of the light spectrum is higher for deoxygenated hemoglobin than for oxygenated hemoglobin. In further embodiments, the emitter(s) 506 may operate and emit light at a wavelength of 940 nm, since the pulsating of blood flow can produce pulsing electrical signals at the photodetector(s) 510. At such wavelengths, the signal strength of pulsating blood can be high so stress assessment (as performed by stress assessment device 602 discussed in FIG. 6) will be accurate.

The sensor 512 can be configured to detect the one or more light signals emitted by the at least one illuminator 508. When more than one illuminator 508 is selected, then each of the illuminator(s) 508 may be paired with a respective photodetector 510 including the sensor(s) 512. The sensor(s) 512 are sensitive to the wavelength range of the respectively paired illuminator(s) 508. The at least one sensor 512 may be paired with the at least one illuminator 508. The at least one sensor 512 can also generate one or more electrical signals based on the one or more light signals.

The sensing device 504 also includes at least one amplifier 514 configured to amplify and filter the one or more electrical signals for enhancing the signal-to-noise ratio (SNR). The sensing device 504 also includes an analog-to-digital convertor 516 configured to convert the amplified and filtered analog signals into one or more digital signals.

In some embodiments, the sensing device 504 operates as a transmissive sensing device or configuration. In the transmissive sensing device, the at least one photodetector 510 measures an intensity of the light that has passed through a chord of living tissue of the agent 102. The light is emitted by the illuminator 508 coupled to the at least one photodetector 510. In alternative embodiments, the sensing device 504 operates as a reflective sensing device or configuration. In the reflective sensing device, the at least one photodetector 510 may measure an intensity of light that has reflected from a surface of the skin of the agent. In such cases, the light may be emitted by the illuminator 508 coupled to the at least one photodetector 510.

In both the configurations, the time-series signal(s) are generated continuously by continuous sensing of the light intensities. In some embodiments, any one pair of the emitter 506 and the photodetector 510 is adequate to obtain the pulsating time-series signal. Further, when the emitters 506 of similar wavelength are selected, then average time-series signal is obtained by averaging the measurement of signals emanating from at least two photodetectors 510 of two pairs of the emitter 506 and the photodetector 510 of similar wavelengths. The time-series signal includes a PPG signal of the subject i.e. the agent 102. In some embodiments, the time-series signal is processed to extract the continuous photoplethysmographic signal. The continuous PPG signal may be analyzed to determine the stress level of the agent 102.

The wearable device 502 also includes an identity generating device 518 configured to generate identification information of the agent wearing the wearable device 502. The identity information may include a name, an agent identity (ID). The identity generating device 518 is also configured to send the generated identification information to the stress assessment device 116. The stress assessment device 116 may correlate the customer interaction data with the determined stress-level based on the identification information associated with the agent.

FIG. 6 is a block diagram 600 illustrating various system elements of an exemplary stress assessment device 602, in accordance with an embodiment of the present disclosure. The stress assessment device 602 can be configured to determine a stress level of the agent 102 based on a processing of at least one PPG signal. The stress assessment device 602 may include one or more processors 604, one or more interfaces 606, and a system memory 608 including an agent identification device 610, a signal analysis device 612, a customer interaction analysis device 614 (or interaction analysis device 614), and a feedback device 616.

The stress assessment device 602, in one embodiment, is a hardware device with at least one processor 604 executing machine readable program instructions for analyzing received videos such that the agent's stress-level can be determined to generate feedback. Such a system may include, in whole or in part, a software application working alone or in conjunction with one or more hardware resources. Such software applications may be executed by the processors on different hardware platforms or emulated in a virtual environment.

The processor(s) 604 may include, for example, microprocessors, microcomputers, microcontrollers, digital signal processors, central processing units, state machines, logic circuits, and/or any devices and computer memory that manipulate signals based on operational instructions. Among other capabilities, the processor(s) 604 are configured to fetch and execute computer readable instructions.

The interface(s) 606 may include a variety of software interfaces, for example, application programming interface; hardware interfaces, for example, cable connectors; or both. As discussed with reference to FIG. 1, the interface(s) 606 may facilitate receiving the video of the region of interest on the agent's body, the customer interaction data, and agent identification data. The interface(s) 606 may further facilitate reliably transmitting feedback to the agent device 108 and/or the supervisor device 122.

The agent identification device 610 stores different types of data to identify each of the agents, such as the agent 102, interacting with the customer 106. Examples of the data include, but are not limited to, employment data (e.g., agent name, agent employee ID, designation, tenure, experience, previous organization, supervisor name, supervisor employee ID, etc.), demographic data (e.g., gender, race, age, education, accent, income, nationality, ethnicity, area code, zip code, marital status, job status, etc.), psychographic data (e.g., introversion, sociability, aspirations, hobbies, etc.), system access data (e.g., login ID, password, biometric data, etc.), and other business-relevant data about each of the agents. Some embodiments may include the agent identification device 610 to store similar data for a supervisor, such as the supervisor 104.

The signal analysis device 612 receives the agent's time-series signal(s) or PPG signal(s) and the corresponding agent identification data from the wearable device 120 directly or indirectly via the agent device 108. In one embodiment, the received time-series signal(s) and/or PPG signal(s) of the agent 102 are processed by the signal analysis device 612 using various techniques known in the art, related art, or developed later. The obtained time-series signal is normalized and filtered to remove undesirable frequencies.

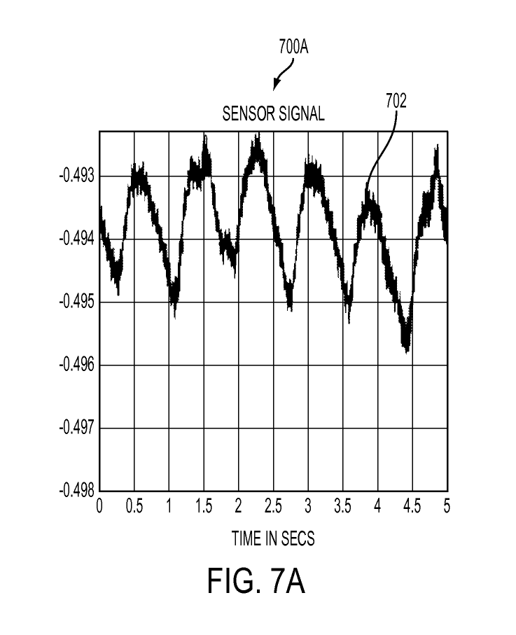

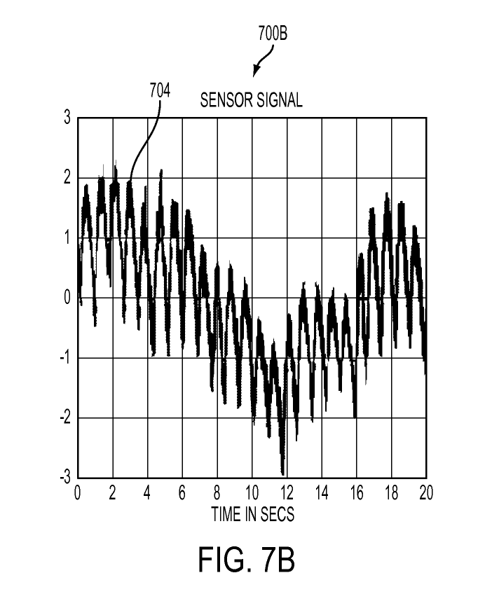

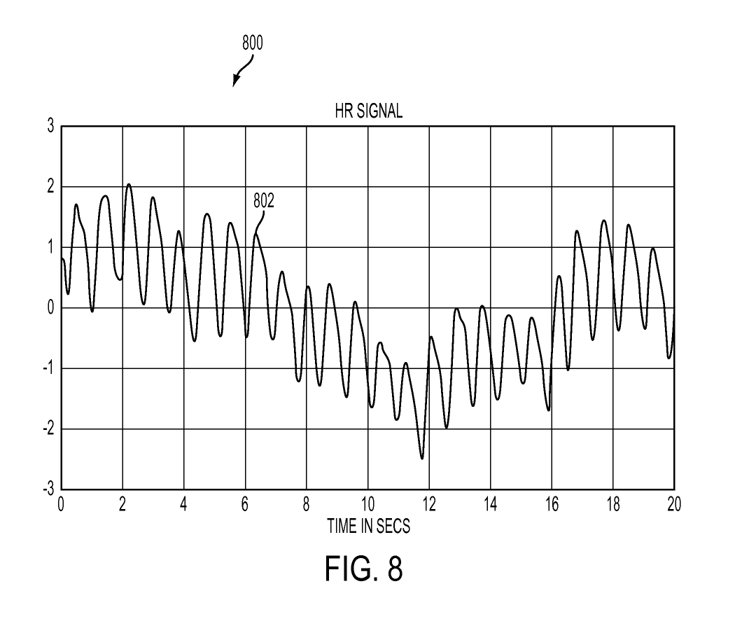

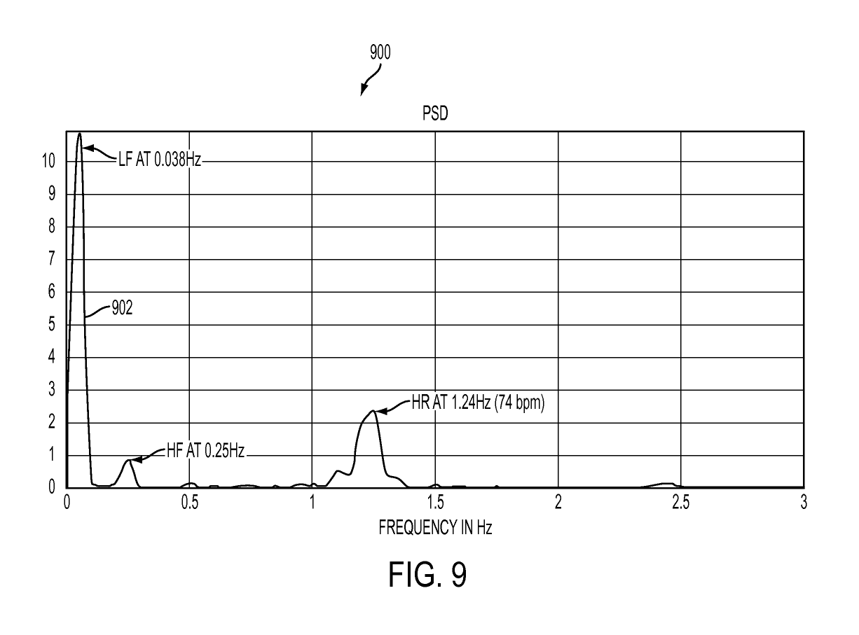

FIG. 7A shows a graph 700A depicting an exemplary time-series signal 702. The time-series signal is first processed to generate photoplethysmographic signal(s) of the subject (or the agent 102) in a continuous fashion using one or more overlapped batches of signals in the time-series signal. The one or more batches of the signals may be created by sliding a window of a desired length, for example, 20 second, in the time-series signal with 96.67% overlap between consecutive batches, which means using only 1 second of new data and retaining 14 second of data from previous batch of signals in the time-series signals or data. Further, to create a continuous pulsatile signal, the extracted pulsatile signal(s) are joined between the successive batches. This may provide an ability to construct a stream of pulsatile signal as in the PPG waveform (see FIG. 9). The signal analysis device 612 may construct the stream of pulsatile signal. Further, the signal analysis device 612 may process the time-series signal of each batch as follows: 1) normalizing the time-series signal by removing a mean and dividing by a standard deviation; and 2) filter the normalized time-series signal to retain the frequency band of interest. The signal analysis device 612 may remove slow, non-stationary frequency components and high frequencies for filtering the normalized time-series signal. FIG. 7B illustrates a graph 700B including an exemplary normalized time-series signal 704 and FIG. 8 is a graph 800 showing an exemplary filtered time-series signal 802 of the agent 102 for a 20 second batch. Further, a bandwidth used for filtering is within a range of 0.01 to 3 Hz.

The resulting time-series signal, i.e., the normalized and filtered time-series signal 802, includes the sum total of volumetric pressure changes within those regions. Arterial pulsations include a dominant component of these signals. The time-series signal includes a PPG signal that correlates to the agent's cardiac pulse pressure wave. The PPG signal may be de-trended to remove slow non-stationary frequency components from the time-series signal such that a nearly stationary PPG signal, and hence a nearly stationary time-series signal, can be obtained.

Referring to FIG. 6, the signal analysis device 612 extracts the low frequency and high frequency components from the time-series signal over a predefined time interval. The signal analysis device 612 also computes a ratio of the low and high frequency (LF/HF ratio) of the integrated power spectrum of the corresponding time-series signal. The LF/HF ratio provides a measure of the agent's estimated HRV for that predefined time interval. The estimated HRV is then used to assess the level of agent stress.

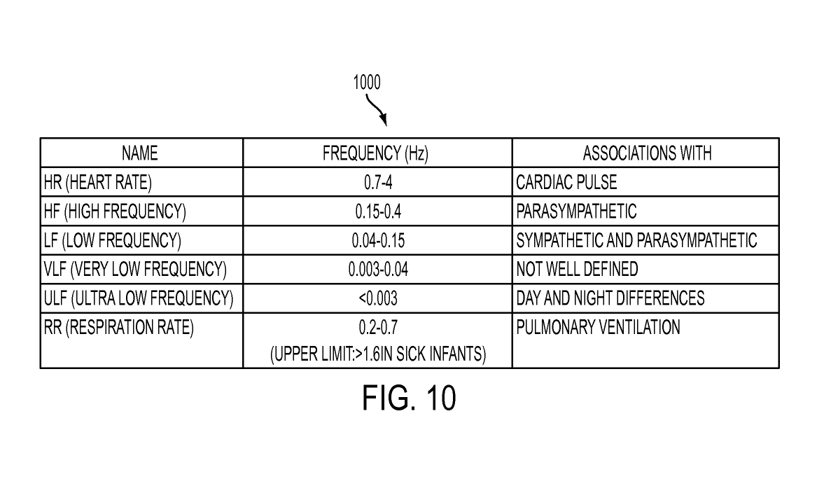

The LF and HF components are related, in different degrees, to different components of the cardio-vascular control system as shown in a table 1000 in FIG. 10. The table 1000 also shows different frequency components for normal healthy humans. The HF component, which has a peak at respiratory frequency, corresponds to respiratory sinus arrhythmia (RSA) and reflects parasympathetic influence on the heart through efferent vagal activity. The LF component, including fluctuations below 0.15 Hz and usually centered at about 0.1 Hz, is mediated by both cardiac vagal and sympathetic nerves. Hence, the LF/HF ratio represents the sympatho-vagal interaction. The LF and HF components may be also expressed in normalized units as shown in Equations (1) and (2) to account for inter-individual differences amongst various LF components and the HF components within their respective frequency ranges. Such normalization of the LF and HF components also normalizes the differences in various pairs of the emitter 506 and the photodetector 510.

$$\begin{matrix}{LF}_{n} = \frac{LF}{Total Power - VLF} & ( 1 ) \\ {HF}_{n} = \frac{HF}{Total Power - VLF} & ( 2 )\end{matrix}$$

In the Equations (1) and (2), the “Total Power” refers to total power of the integrated spectrum containing the LF and HF Components over the predefined time interval within the time-series signal; and “VLF” refers to a very low frequency ranging from 0.003 Hz to 0.04 Hz over the predefined time interval within the time-series signal.

In an embodiment, the LF/HF ratio exceeding a threshold value of “1” indicates abnormal stress-level of the agent 102. The signal analysis device 612 may include multiple stress threshold values that are compared to the computed value of LF/HF ratio for determining the level of agent stress. For example, a value of the LF/HF ratio between stress threshold values “1” to “2” may indicate low-level stress. Similarly, a value of the LF/HF ratio between the stress threshold values “2” and “3” may indicate mid-level stress, and that between the stress threshold values “3” and “4” may indicate high-level stress experienced by the agent 102. The LF/HF ratio having a value less than the stress threshold value “1” corresponds to the influence of PNS indicating insignificant or normal-level agent stress.

Such non-contact estimation of HRV based on analyses of agent's time-series signal/data to determine agent stress does not involve active involvement of the agent 102, thereby minimizing the chances of agent pretense.

The customer interaction analysis device 614 receives the customer interaction data and the agent identification data from the agent device 108. The customer interaction analysis device 614 is configured to analyze the customer interaction data including at least one of customer-related actions including: (1) the customer's responses to the IVR system; (2) the text messages, the audio interactions, and the video interactions exchanged between the customer 106 and the agent 102 (or the supervisor 104); (3) customer-provided feedback; and (4) a customer call summary created by the agent 102 based on the agent's interaction with the customer 106, or in any combination thereof. In one example, the customer interaction analysis device 614 may apply Automatic Speech Recognition (ASR) on the agent-customer conversation followed by text analysis of the ASR transcript to parse the conversation into different categories. The categorization may be performed on the basis of different parameters such as modeling the customer call flow (for example, which part of the call was “greeting”, “query”, “closing” and so on), spotting key words or generic terms of interest (e.g., 16-character alphanumeric customer ID, 10-digit phone number), sentiment-intensive words (e.g., “hate”, “irritate”, etc.), customer-agent voice-over (indicative of impatience and irritation on customer's side) and other finer aspects. In another example, the customer interaction analysis device 614 may parse the customer's IVR responses to identify various aspects such as a broad topic of a customer call, the caller's state of mind (i.e., is the customer 106 agitated, in a hurry or calm), customer interaction history (i.e., number of times the customer 106 has called in the recent past, how much time the customer 106 has spent in traversing the IVR) and in many cases a fine grain sub-topic identification. The customer interaction analysis device 614 can identify these parameters and aspects as the stress-trigger points responsible for causing agent tress.

Further, the customer interaction analysis device 614 is configured to combine the customer interaction data with the agent stress profile determined by the signal analysis device 612 based on the agent identification data. For example, customer interaction analysis device 614 may correlate various customer-related actions with the time of high agent stress based on the agent login ID to derive insights into the stress-trigger points for the agent 102. In the embodiment, stress trigger points can include (a) the agent 102 is more stressed during a ‘query’ period (such that a likely implication is that the access to database is slow or the script to identify a customer issue is not easy to follow) or during a “resolution” period (such that a likely implication is that a manual provided for troubleshooting is not detailed enough or is faulty and needs revision); (b) the agent 102 is stressed by customer's language and tone (such that a likely implication is that the agent 102 needs training on how to empathize with the customer 106 or that the call should be escalated); or (c) the agent 102 is highly stressed throughout the call (such that a likely implication is that the agent 102 has difficulty following a particular accent of the customer 106 or is not well versed in a particular customer-related topic). Such insights are also identified as the stress-trigger points for the agent 102 by the customer interaction analysis device 614. The customer interaction analysis device 614 communicates the stress-trigger points to the feedback device 616.

In the embodiments, feedback device 616 receives the stress-trigger points and is configured to provide feedback to at least one of the agent 102 and the supervisor 104 based on outputs of the signal analysis device 612 and the customer interaction analysis device 614. In one example, the feedback device 616 may generate feedback whenever the signal analysis device 612 determines that the LF/HF ratio has exceeded one or more predefined stress threshold values indicating a stress pattern of the agent 102. In another example, predefined suggestive remedial messages may be stored in the feedback device 616. The predefined suggestive remedial messages may be created based on the identified stress-trigger points. The feedback device 616 may be configured to generate feedback including the predefined suggestive remedial messages to assist in reducing stress of the agent 102.

Additionally, the identified stress-trigger points may be retrieved from the feedback device 616 upon request. The retrieved stress-trigger points may be used by the supervisor 104 or the agent 102 offline for various purposes, such as to identify a set of agents that may be best suited for a particular customer-related topic, the agents who need further training on particular customer-related topics, and those customer-related topics that need better training material.

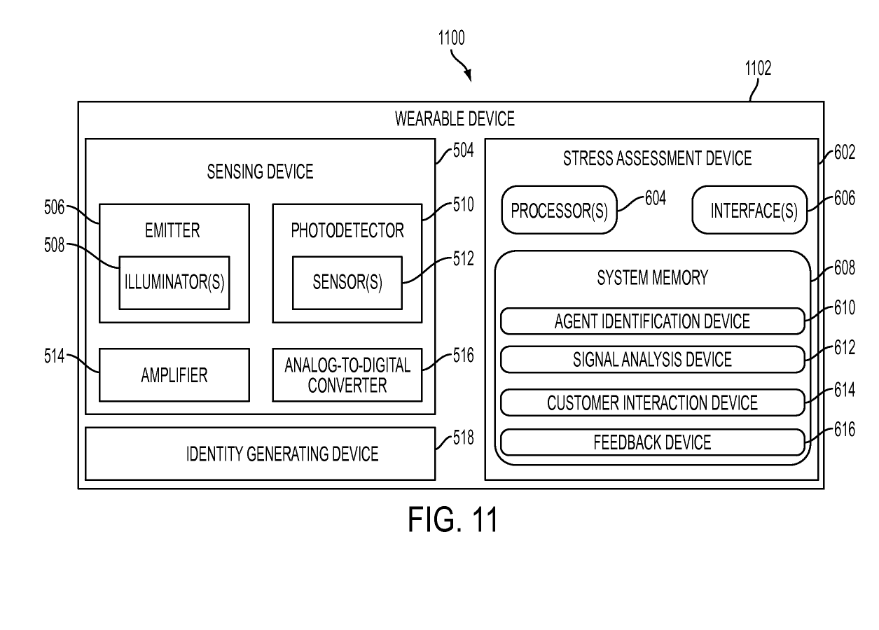

In some embodiments, the wearable device 502 may include the one or more components of the stress assessment device 602. FIG. 11 illustrates a block diagram 1100 of an alternative wearable device 1102 including the devices of the stress assessment device 602 that is described above relating to FIG. 6.

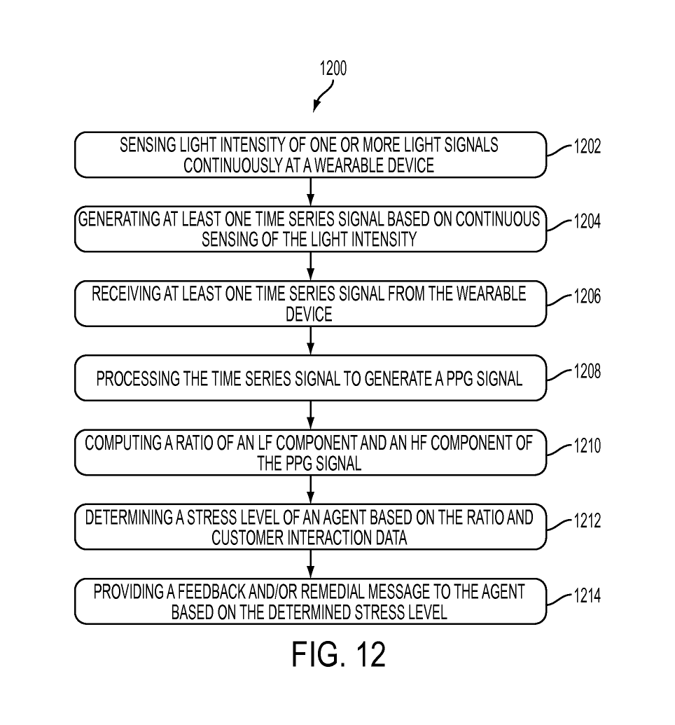

An exemplary method 1200 for assessing and managing the stress of an agent, such as the agent 102, according to an embodiment of the present disclosure is illustrated in the flowchart of FIG. 12. As discussed with reference to FIG. 1, the agent 102 communicates with the customer 106 and may get stressed during the communication due to various reasons. To estimate a stress level of the agent 102, the wearable device 120 and the stress assessment device 116 of the embodiments may be used. The exemplary method may be described in the general context of computer executable instructions. Generally, computer executable instructions can include routines, programs, objects, components, data structures, procedures, devices, functions, and the like that perform particular functions or implement particular data types. The computer executable instructions can be stored on a computer readable medium, and installed or embedded in an appropriate device for execution.

The order in which the method is described is not intended to be construed as a limitation, and any number of the described method blocks can be combined or otherwise performed in any order to implement the method, or an alternate method. Additionally, individual blocks may be deleted from the method without departing from the spirit and scope of the present disclosure described herein. Furthermore, the method can be implemented in any suitable hardware, software, firmware, or combination thereof, that exists in the related art or that is later developed.

The method 1200 describes, without limitation, implementation of the exemplary stress assessment device 116 and the wearable device 120 in a call center environment. While the embodiments are described implemented at a call center, the implementation in a specific business or service is not limiting. One of skill in the art will understand that the systems, devices, and methods of the embodiments may be modified appropriately for implementation in a variety of other business scenarios including those related to medical services, hospitality, retail, banking services, and so on, without departing from the scope and spirit of the disclosure.

At step 1202, light intensity of one or more light signals that are illuminated by at least one illuminator 508 of the wearable device 502 is sensed at the wearable device 502. In some embodiments, the sensor associated with the photodetector 510 in the wearable device 502 senses the light intensity. As the agent 102 interacts with the customer 106 during or after the conversation, the agent 102 may experience stress for various reasons such as agent may feel stressed while dealing with irate customer 106. To continuously assess the stress of the agent 102, he/she may wear the wearable device 502 on an area of exposed skin of the agent 102. The wearable device 502 includes one or more pairs of the emitter 506 and the photodetector 510. The illuminator 508 of the emitter 506 emits the light, which can be sensed by the sensor 512 in some embodiments. The pair of the emitter 506 and the photodetector 510 may be present on an inner side of the wearable device 502. In some embodiments, the emitter(s) 506 may be set to emit source light at a wavelength range centered on 660 nm because the absorbance of light in the red region of the light spectrum is higher for deoxygenated hemoglobin than for oxygenated hemoglobin. In alternative embodiments, the emitter(s) 506 may emit light at a wavelength of 940 nm since the pulsating blood flow of agent 102 can produce pulsing electrical signals at the photodetector 510. Further, signal strength of pulsating blood will be high at these wavelength ranges of its respective paired illuminator 508. In some embodiments, the electrical signal(s) from the photodetector 510 may be amplified and filtered by the amplifier 514 to enhance signal-to-noise (SNR) ratio. The amplified and filtered analog signal is converted to digital signal by the high-resolution analog-to-digital converter 516. The digital signal is then subjected to further data processing.

At step 1204, at least one time-series signal is generated based on the sensed intensity of light. In some embodiments, the sensor 512 generates the time-series signal at the wearable device 502. The time-series signal may include at least one PPG signal of the subject, such as the agent 102. Then, at step 1206, the wearable device 502 may send/transmit the at least one time-series signal to the stress assessment device 602 of FIG. 6 for further processing.

In the embodiments, the stress assessment device 602 may also receive the customer interaction data from the wearable device 502. Further, at step 1208, the signal analysis device 612 processes the received time-series signal to generate a PPG signal from the time-series signal of the agent 102. The signal can be processed over a time period and can be processed in continuous fashion using overlapped batches. The batches may be created by sliding a window of a desired length say, 20 seconds, with 96.67% overlap between consecutive batches, which means using only 1 second of new data and retaining 14 seconds of data from previous batch. To create a continuous pulsatile signal, the signal analysis device 612 may join or stitch the extracted pulsatile signal between the successive batches. This provides the abilities to construct a stream of pulsatile signal as in the PPG waveform. The time-series signal of each batch is processed as follows: the time-series signal is normalized by removing the mean and dividing by the standard deviation, then the normalized signal is filtered to retain the frequency band of interest (i.e., remove slow non-stationary frequency components and high frequencies). The resultant signal is called the PPG signal. Sensor time-series signal for the agent 102 is shown in FIG. 7A. FIG. 7B and FIG. 8 shows signals after normalization and band pass filtering, respectively, for 20 second batch.

Then, at step 1210, a ratio of a Low Frequency (LF) component and a High Frequency (HF) component of the PPG signal is computed. Heart Rate Variability (HRV) is a common measure that may be used for evaluating stress of the agent 102 by determining state of the autonomic nervous system (ANS) using continuous pulsatile signal as described above. The ANS is represented by the sympathetic nervous system (SNS) and the parasympathetic nervous system (PNS). The HRV is the beat-to-beat time variation in heartbeat and is modulated by changes in the balance between influences of the SNS and the PNS. Such changes occur based on the response of the agent's body to stress by releasing hormones, such as epinephrine and cortisol, which in turn lead to increase in heartbeats, tightening of muscles, and increase in blood pressure. The HRV is also useful for diagnosis of various diseases and health conditions such as diabetic neuropathy, cardio vascular disease, myocardial infraction, fatigue, sleep problems, psychiatric disorders, psychological disorders, etc.

There are numerous methods (e.g., time domain, frequency domain) available for analyzing bio signal time-series (or PPG) described above. Time domain methods use analyzing peak to peak pulse intervals or sequence of intervals between successive fiducial points in the pulsatile signal. More accurate time domain methods involve use of peak to peak intervals from the p-wave of the ECG because it actually measures the rhythms associated with the sinoatrial node which is the pacemaker of the heart. In normal subjects, such as the agent 102, the HRV measured from the pulsatile signal (time-series signal) is considered adequate for estimating the stress level. Most common time domain estimate of the HRV is the standard deviation of peak to peak interval (SDNN), i.e., normal to normal deviation when measured between consecutive sinus beats. Other time domain measures such as RMSSD (root mean square of successive differences of peak to peak intervals), the number of pairs of adjacent peak to peak intervals differing by more than 50 ms (NN50 count) are also used. However, these metrics primarily provide high frequency (HF) variations of PPG signal. Low Frequency (LF) variations are important, but cannot be easily obtained from time domain methods.

Since the PPG signal contains many well-defined rhythms (LF b/w 0.04 to 0.15 Hz; HF b/w 0.15 to 0.4 Hz; HR b/w 0.7 to 4Hz and harmonics of these components), the disclosed stress assessment device 602 may use frequency domain methods that extract the ratio of powers in low-frequency propose (LF) and high-frequency (HF) components of the power spectral density (PSD) generated from the PPG signal. PSD function contains distribution of frequency components. The signal analysis device 612 may compute an area under the PSD curve corresponding to 0.04-0.15 Hz and 0.15-0.4 Hz. The area may respectively provide values of the LF and HF components. When the LF/HF ratio is greater than a stress threshold value, for example, a value greater than “1”, the agent's SNS is more dominant and hence indicates that the agent 102 is under stress. FIG. 9 shows the LF and HF components along with heart rate frequency. FIG. 9 shows the power spectrum of photoplethysmographic signal. The HF component corresponds to heart rate variations related to the respiratory sinus arrhythmia (i.e., respiratory frequency) and are mediated by parasympathetic activity. LF component is associated with activities in both sympathetic and parasympathetic nervous system. The LF and HF components may be also expressed in normalized units as shown in Equations (1) and (2) described above to account for inter-individual differences amongst various LF components and the HF components within their respective frequency ranges. Note, proper analysis involves using the recommended batch length of 5 minutes or longer. For convenience, in this test, we collected time-series signal for only 20 seconds.

At step 1212, a stress level of the agent 102 is determined based on the processing of the time-series signal (or time-series data) and the customer interaction data. In some embodiments, the customer interaction data is analyzed by the customer interaction analysis device 614 and the stress level of the agent 102 is determined by the signal analysis device 612. The various device of the stress assessment device 602 may communicate with each other or may exchange information with each other. The customer interaction data may be analyzed based on various parameters as described above.

Thereafter, at step 1214 a feedback or a remedial message is generated and provided to the agent 102 that is at least partially based on the determined stress level of the agent 102. In some embodiments, the feedback is generated by the feedback device 616. The feedback device 616 may assess an increasing level of the agent's stress based on the increasing LF/HF ratio with respect to multiple predefined stress thresholds. Based on the assessment, the signal analysis device 612 may create a stress profile for the agent 102. Whenever the LF/HF ratio exceeds each of the predefined stress thresholds, the feedback device 616 may generate the feedback. Such stress profiles of different agents may also be used, such as by the supervisor for various purposes. For example, the agent stress profile may assist the supervisor to identify a set of agents that may be appropriate for: (1) a particular customer concern or issue; (2) further training on particular customer concerns or issues; (3) customer concerns or issues that need better training material for the agents, and so on. This data can be combined with audio and video system (when available) in which customer's audio responses to the agent, color or textual changes in the exposed skin area of the agent, etc., to further improve/enhance the diagnosis/performance of stress assessment system. In one embodiment, the feedback device 616 is configured to receive the agent stress profile from the signal analysis device 612. When the agent stress profile indicates that the LF/HF ratio exceeds a predefined stress threshold value, such as, “1”, “2”, and so on, the feedback device 616 is configured to provide feedback to the corresponding agent 102 or to the supervisor 104 in real-time. Other embodiments may include the feedback device 616 is configured to provide the feedback to the agent 102 or the supervisor 104 multiple times if the LF/HF ratio is equivalent to or above one or more predefined stress threshold values for a predetermined time. Additionally or alternatively, the feedback device 616 may be configured to provide the feedback to the agent 102 or the supervisor 104 when the LF/HF ratio reduces below one or more predefined stress threshold values.

In the various embodiments, the feedback may be provided in various forms including, but not limited to, an alert message, an audio indication such as a beep, and a visual indication such as a blinking light, or any combination thereof. In other embodiments, feedback can be provided from feedback device 616 to the agent 102 through any method or device that can provide the remedial message or feedback based on the determined stress level. The feedback provides an audible or passive indication to the agent 102 that the agent is experiencing stress while interacting with the customer 106. For example, when the agent 102 is communicating with an irate customer 106, the agent 102 may experience abnormal stress. Upon detecting agent stress due to an increase in the LF/HF ratio beyond “1” as indicated in the agent stress profile, the feedback device 616 provides the feedback to the agent 102 and the supervisor 104 in real-time during a live customer interaction. In exemplary embodiments including those involving non-visual interactions, such as voice calls, between the agent 102 and the customer 106 in a live environment, the provided feedback provides an indication to the agent 102 the need to reduce stress and that the agent may need to change the course of interaction. The stress assessment device can be worn for longer period to monitor agent's physiological state further and reinstate his/her activities after the stress level reaches the desired state. In some embodiments, the feedback device 616 may provide the feedback to the supervisor 104 on the supervisor device 122 during an on-going customer-agent interaction. As a result, the supervisor 104 is able to effectively monitor multiple customer-agent interactions and provide relevant assistance to the agent 102 for ensuring or enhancing customer satisfaction.

In an alternative embodiment, the feedback may be combined with suggestive stress-remedial messages based on the identified stress-trigger points. In one embodiment, the feedback device 616 is configured to provide predefined stress remedial messages with the feedback to the agent 102 based on the received stress-trigger points. The predefined stress remedial messages may assist the agent 102 during the live customer interaction to reduce agent stress. For example, the agent 102 may be experiencing stress due to lack of adequate information to address customer 106 needs. In response to an increasing agent stress, the feedback device 616 may provide the agent 102 with a link to a technical guide along with stress-indicating feedback to assist the agent 102 on-the-fly to successfully address the customer 106 needs and reduce agent stress. In another example, if the agent stress-level remains above the predefined stress threshold for a predefined duration during the customer call, the supervisor 104 may be alerted by sending feedback or the agent 102 may be prompted to escalate the call. Unlike the conventional offline analyses of the customer interaction data, the feedback device 616 analyzes the stress-trigger points to provide real-time appropriate feedback message during a live customer-agent interaction for mitigating agent stress and enhance customer satisfaction.

Other embodiments may include the feedback device 616 configured to provide on-demand stress-related data for each agent 102 for non-continuous monitoring of customer-agent interactions. In one example, the supervisor 104 may request to review the stress-trigger points for a particular agent 102 during various customer calls to perform an aggregate-level stress analysis for the agent 102. In another example, the supervisor 104 may request stress-trigger points for a set of agents to identify high stress times and high stress agents for managing deputation of one or more supervisors. Such analysis of the stress-related data may assist the supervisor 104 to monitor long-term performance of one or more agents to improve call routing and to improve various resources, such as training, learning material, number of breaks, allocated projects of a particular type, etc., available to the agents.

The stress assessment device 602 with the body-worn wearable device 502 and analysis and interaction devices described above correlate the analyzed customer interaction data to identify stress-trigger points.

The above description does not provide specific details of manufacture or design of the various components. Those of skill in the art are familiar with such details, and unless departures from those techniques are set out, techniques, known, related art or later developed designs and materials should be employed. Those in the art are capable of choosing suitable manufacturing and design details.

The terminology used herein is for the purpose of describing particular embodiments only and is not intended to be limiting of the disclosure. It will be appreciated that several of the above-disclosed and other features and functions, or alternatives thereof, may be combined into other systems, methods, or applications. Various presently unforeseen or unanticipated alternatives, modifications, variations, or improvements therein may subsequently be made by those skilled in the art without departing from the scope of the present disclosure as encompassed by the following claims.

What is claimed is:

1. A system for assessing and managing stress of an agent while interacting with a customer, the system comprising:
a wearable device configured to be worn by the agent, the wearable device comprises a sensing device configured to generate at least one time-series signal by continuous sensing of light intensity of one or more light signals, the at least one time-series signal comprises at least one continuous photoplethysmographic (PPG) signal including at least one low frequency (LF) component and at least one high frequency (HF) component; and
a stress assessment device using a processor to determine a stress level of the agent based on a processing of the at least one PPG signal, wherein the stress assessment device further comprises:
a signal analysis device configured to:
receive the at least one time-series signal from the wearable device;
process the at least one time-series signal to generate the at least one continuous PPG signal;
compute a ratio of the at least one LF component and the at least one HF component;
determine the stress level of the agent based on the ratio exceeding a predefined stress threshold; and
generate a stress profile of the agent based on the determined stress level;
a customer interaction analysis device configured to:
analyze customer interaction data of the agent based on a plurality of parameters; and
determine stress-trigger points from the customer interaction data for the agent; and
a feedback device to provide a feedback comprising at least one remedial message to the agent based on the determined stress level of the agent.

2. The system of claim 1, wherein the customer interaction data includes at least one of customer responses to an interactive voice response (IVR) system, audio interactions, video interactions, text messages, call summaries, and customer-provided feedback, wherein the audio interactions include at least one of voice calls and recorded audio messages, and the video interactions include at least one of video calls and recorded video messages.

3. The system of claim 3, wherein the customer interaction analysis device is configured to:
apply Automatic Speech Recognition (ASR) on the customer interaction data to generate at least one ASR transcript; and
analyze text of the ASR transcript to parse the conversation into one or more categories.

4. The system of claim 1, wherein the signal analysis device is further configured to determine the stress level of the agent based on a correlation between the customer interaction data and the determined stress-level over a predefined time interval.

5. The system of claim 1, wherein the sensing device further comprises:
at least one emitter fixed to an inner surface of the wearable device, wherein the emitter comprises at least one illuminator configured to emit light at a specified wavelength band; and
at least one photodetector coupled to the emitter, the photodetector further comprises at least one sensor paired with the at least one illuminator, the at least one sensor is configured to:
detect the one or more light signals emitted by the at least one illuminator, wherein the at least one sensor is sensitive to the wavelength of range of the corresponding illuminator; and
generate one or more electrical signals based on the one or more light signals;
at least one amplifier configured to amplify and filter the one or more electrical signals for enhancing the signal-to-noise ratio; and
an analog-to-digital convertor configured to convert the amplified and filtered analog signals into one or more digital signals.

6. The system of claim 5, wherein the sensing device is a transmissive sensing device, wherein the at least one photodetector measures an intensity of the light that has passed through a chord of living tissue of the agent, wherein the light is emitted by the illuminator coupled to the at least one photodetector.

7. The system of claim 5, wherein the sensing device is a reflective sensing device, wherein the at least one photodetector measures an intensity of light that has reflected off a surface of the skin of the agent, wherein the light is emitted by the illuminator coupled to the at least one photodetector.

8. The system of claim 1, wherein the generated feedback includes at least one of a real-time message, at least one predefined stress remedial message, an audio indication, and a visual indication for the agent.

9. The system of claim 1, wherein the wearable device further comprises an identity generating device configured to:
generate an identification information of the agent wearing the wearable device; and
send the generated identification information to the stress assessment device, wherein the customer interaction data is correlated with the determined stress-level based on the identification information associated with the agent.

10. A method for assessing and managing stress of an agent while interacting with a customer, the method comprising:
generating, by a sensing device of a wearable device, at least one time-series signal based on continuous sensing of light intensity of the one or more light signals, the agent wears the wearable device during the sensing, wherein the at least one time-series signal comprises at least one photoplethysmographic (PPG) signal including at least one low frequency (LF) component and at least one high frequency (HF) component;
determining, by a stress assessment device, a stress level of the agent based on a processing of the at least one PPG signal in real time by:
processing, by a signal analysis device, the at least one time-series signal to generate the at least one continuous PPG signal;
computing, by the signal analysis device, a ratio of the at least one LF component and the at least one HF component;
determining, by the signal analysis device, the stress level of the agent based on the ratio exceeding a predefined stress threshold; and
generating, by the signal analysis device, a stress profile of the agent based on the determined stress level; and
providing, by a feedback device, a feedback to the agent based on the determined stress level of the agent in the real time.

11. The method of claim 10, wherein the predefined stress threshold has one or more values set as a threshold above which the agent becomes stressed due to customer interaction.

12. The method of claim 10, wherein the customer interaction data includes at least one of customer responses to an interactive voice response (IVR) system, audio interactions, video interactions, text messages, call summaries, and customer-provided feedback, wherein the audio interactions include at least one of voice calls and recorded audio messages, and the video interactions include at least one of video calls and recorded video messages.

13. The method of claim 10 further comprising:
applying, by the customer interaction analysis device, Automatic Speech Recognition (ASR) on the customer interaction data to generate at least one ASR transcript; and
analyzing, by the customer interaction analysis device, text of the ASR transcript to parse the conversation into one or more categories.

14. The method of claim 10 further comprising determining, by the signal analysis device, the stress level of the agent based on a correlation between the customer interaction data and the determined stress-level over a predefined time interval.

15. The method of claim 10 further comprising:
emitting, by at least one illuminator of at least one emitter, the light at a specified wavelength, wherein the at least one emitter is fixed to an inner surface of the wearable device;
detecting, by at least one sensor of a photodetector coupled to the at least one emitter, the one or more light signals emitted by the at least one illuminator, wherein the at least one sensor is sensitive to the wavelength of range of the corresponding illuminator;
generating, by the sensor, one or more electrical signals based on the one or more light signals;
amplifying and filtering, by at least one amplifier of the sensing device, the one or more electrical signals for enhancing the signal-to-noise ratio; and
converting, by an analog-to-digital convertor of the sensing device, the amplified and filtered analog signals into one or more digital signals.

16. The method of claim 10 further comprising measuring, by the at least one photodetector, an intensity of the light which has passed through a chord of living tissue of the agent, wherein the light is emitted by the illuminator coupled to the photodetector.

17. The method of claim 10 further comprising measuring, by the at least one photodetector, measures an intensity of light which has reflected off a surface of the skin of the agent, wherein the light is emitted by the illuminator coupled to the at least one photodetector.

18. The method of claim 10 further comprising:
generating, by an identity generating device of the wearable device, identification information of the agent wearing the wearable device; and
sending, by the identity generating device, the generated identification information to the stress assessment device, wherein the customer interaction data is correlated with the determined stress-level based on the identification information associated with the agent.

19. A system for assessing and managing stress of a user, the system comprising:
a wearable device configured to be worn by the user, the wearable device comprises a sensing device for generating at least one time-series signal by continuous sensing of light intensity of one or more light signals, the at least one time-series signal comprises at least one continuous photoplethysmographic (PPG) signal including at least one low frequency (LF) component and at least one high frequency (HF) component; and
a stress assessment device using a processor to determine a stress level of the user based on a processing of the at least one PPG signal, wherein the stress assessment device further comprises:
a signal analysis device configured to:
process the at least one time-series signal to generate the at least one continuous PPG signal;
compute a ratio of the at least one LF component and the at least one HF component;
determine the stress level of the user based on the ratio exceeding a predefined stress threshold; and
generate a stress profile of the user based on the determined stress level;
an interaction analysis device to:
analyze interaction data of the user based on a plurality of parameters; and
determine one or more stress-trigger points from the interaction data for the user; and
a feedback device to provide a feedback comprising at least one remedial message to the user based on the determined stress level of the user.

20. A method for assessing and managing stress of a user, the method comprising:
generating, by a sensing device of a wearable device, at least one time-series signal based on continuous sensing of light intensity of the one or more light signals, the user wears the wearable device during the sensing, wherein the at least one time-series signal comprises at least one photoplethysmographic (PPG) signal including at least one low frequency (LF) component and at least one high frequency (HF) component;
determining, by a stress assessment device, a stress level of the agent based on a processing of the at least one PPG signal in real time by:
processing, by a signal analysis device, the at least one time-series signal to generate the at least one continuous PPG signal;
computing, by the signal analysis device, a ratio of the at least one LF component and the at least one HF component;
determining, by the signal analysis device, the stress level of the user based on the ratio exceeding a predefined stress threshold;
generating, by the signal analysis device, a stress profile of the user based on the determined stress level;
analyzing, by an interaction analysis device, interaction data of the user base don a plurality of parameters;
determining one or more stress-trigger points from the interaction data for the user; and
providing, by a feedback device, a feedback to the agent based on the determined stress level of the user in the real time.

21. A wearable device for generating feedback to an agent during communication with a customer, the wearable device comprising:
a signal analysis device for determining a stress level of the agent based on the time-series signal received from the wearable device, the wearable device being configured to be worn onto or near an area of exposed skin of the agent, wherein the wearable device is configured to generate at least one time-series signal by continuous sensing of light intensity of one or more light signals, wherein the at least one time-series signal comprises at least one photoplethysmographic (PPG) signal including at least one low frequency (LF) component and at least one high frequency (HF) component;
an interaction analysis device for receiving data collected based communication between the agent and the customer, the interaction analysis device is configured to analyze customer interaction data of the agent and correlate the data with a determined stress-level over the predefined time interval; and
a feedback device for generating feedback to the agent based on the determined stress-level exceeding a predefined stress threshold, the feedback including predefined suggestive messages based on the correlated data.

22. A method for generating feedback to an agent during communication with a customer, the wearable device comprising:
determining, by a signal analysis device, a stress level of the agent based on the time-series signal received from the wearable device, the wearable device being configured to be worn onto or near an area of exposed skin of the agent, wherein the wearable device is configured to generate at least one time-series signal by continuous sensing of light intensity of one or more light signals, wherein the at least one time-series signal comprises at least one photoplethysmographic (PPG) signal including at least one low frequency (LF) component and at least one high frequency (HF) component;
receiving, by an interaction analysis device, data collected based communication between the agent and the customer, the customer interaction analysis device is configured to analyze customer interaction data of the agent and correlate the data with a determined stress-level over the predefined time interval; and
generating, by a feedback device, feedback to the agent based on the determined stress-level exceeding a predefined stress threshold, the feedback including predefined suggestive messages based on the correlated data.
